(19) United States
(12) Patent Application Publication
(10) Pub. No.: US 2024/0216371 A1
(43) Pub. Date: Jul. 4, 2024

(54) HAIR GROWTH COMPOSITION AND METHOD FOR MAKING AND USING THE SAME

(71) Applicant: Camille Bisou Beauty LLC, Zionsville, IN (US)

(72) Inventor: Neema Mayenga, Zionsville, IN (US)

(21) Appl. No.: 18/149,916

(22) Filed: Jan. 4, 2023

(51) Int. Cl. A61K 31/506; A61K 9/00; A61K 31/4188; A61K 36/42; A61K 36/53; A61K 36/534; A61K 36/58; A61K 36/61; A61K 36/886; A61K 36/8962; A61K 36/899

(52) U.S. Cl. CPC A61K 31/506; A61K 9/0014; A61K 31/4188; A61K 36/42; A61K 36/53; A61K 36/534; A61K 36/58; A61K 36/61; A61K 36/886; A61K 36/8962; A61K 36/899

(57) ABSTRACT

A hair regrowth composition including in combination one part by weight 5% solution of 2,4-diamino-6-piperidinopyrimidine and one part by weight admixture. The admixture further includes one part by weight biotin, one part by weight rosemary oil, one part by weight tea tree oil, one part by weight peppermint oil, and one part by weight pumpkinseed oil.

### TECHNICAL FIELD

The invention relates generally to the field of cosmetics, and more specifically to a composition for urging hair growth and method of using the same.

### BACKGROUND

Hair loss currently plagues a great many men and women across the world. While there are certain chemicals that promote hair growth (and regrowth), they do not work very well and are poorly understood. For instance, 2,4-diamino-6-piperidinopyrimidine 3-oxide is a hypertension medication that, when applied topically, is known to promote limited hair growth in people suffering from androgenic alopecia. It is currently unclear as to the mechanism for the hair growth stimulation effects of 2,4-diamino-6-piperidinopyrimidine. Other hair growth preparations include essential oils, herbal concoctions, and the like. However, none of these formulations work particularly well to promote hair growth.

Thus, there remains a need for a hair growth stimulant that works quickly and effectively. The present novel technology addresses this need.

### DETAILED DESCRIPTION OF THE PREFERRED EMBODIMENTS

For the purposes of promoting an understanding of the principles of the claimed technology and presenting its currently understood best mode of operation, reference will now be made to the embodiments and specific language will be used to describe the same. It will nevertheless be understood that no limitation of the scope of the invention is thereby intended, with such alterations and further modifications in the illustrated device and such further applications of the principles of the claimed technology as illustrated therein being contemplated as would normally occur to one skilled in the art to which the claimed technology relates.

In one embodiment of the present novel technology, a hair growth stimulant formula is prepared as follows. Five parts by weight of as 5% solution of 2,4-diamino-6-piperidinopyrimidine are mixed with one part by weight biotin and one part by weight of each of the following oils to yield a homogeneous solution: rosemary oil, tea tree oil, peppermint oil and pumpkinseed oil. One or two drops of the resulting solution are applied topically to the affected area once or twice a day, being careful to not contact the oil with any portions of the skin where hair growth is unwanted. Hair regrowth should be observed within a few days. The above may be varied by increasing or decreasing the amount of one or more ingredients by a factor of two.

Herein, biotin is provided in a glycerin and/or coconut oil carrier component and is standardized to a concentration of 30000 mcg/ml.

In a second embodiment, the following oils and like ingredients are mixed according to the weights given in Table 1.

TABLE 1

| Oil name | Grams | | Ounces | | Per 2 ounces bottles | |
|---|---|---|---|---|---|---|
| Avocado Oil | 283 | g | 10 | oz | 0.3 | oz |
| Rice Bran Oil | 283 | g | 10 | oz | 0.3 | oz |
| Castor Oil | 283 | g | 10 | oz | 0.3 | oz |
| Pumpkin Seed oil | 283 | g | 10 | oz | 0.3 | oz |
| Extra Virgin | 283 | g | 10 | oz | 0.3 | oz |
| Olive Oil | | | | | | |
| Grapeseed Oil | 283 | g | 10 | oz | 0.3 | oz |
| Biotin | 170.1 | g | 6 | oz | 0.2 | oz |
| Chebe Powder | 170.1 | g | 6 | oz | 0.2 | oz |
| Rosemary | 10 | g | 0.4 | oz | 0.013 | oz |
| Aloe vera | 10 | g | 0.4 | oz | 0.013 | oz |
| Fenugreek | 9.4 | g | 0.3 | oz | 0.01 | oz |
| Rose Petals | 9.4 | g | 0.3 | oz | 0.01 | oz |
| Neem | 5.5 | g | 0.19 | oz | 0.006 | oz |
| Bhringraj | 5.5 | g | 0.19 | oz | 0.006 | oz |
| Cloves | 5.5 | g | 0.19 | oz | 0.006 | oz |
| Curry Leaves | 5.5 | g | 0.19 | oz | 0.006 | oz |
| Poolankilangu | 5.5 | g | 0.19 | oz | 0.006 | oz |
| Karboga Arisi | 5.5 | g | 0.19 | oz | 0.006 | oz |
| Vembalam Pattai | 5.5 | g | 0.19 | oz | 0.006 | oz |
| Kadukkai | 5.5 | g | 0.19 | oz | 0.006 | oz |
| Avarampoo | 5.5 | g | 0.19 | oz | 0.006 | oz |
| Maruthani Vithai | 5.5 | g | 0.19 | oz | 0.006 | oz |
| Samangi Vithai | 5.5 | g | 0.19 | oz | 0.006 | oz |
| Nellikai | 5.5 | g | 0.19 | oz | 0.006 | oz |
| Magilampoo | 5.5 | g | 0.19 | oz | 0.006 | oz |
| Avarakkai | 5.5 | g | 0.19 | oz | 0.006 | oz |
| Velankuchi Ver | 5.5 | g | 0.19 | oz | 0.006 | oz |
| Onion | 5.5 | g | 0.19 | oz | 0.006 | oz |
| Karunjeeragam | 5.5 | g | 0.19 | oz | 0.006 | oz |
| Vasambu | 5.5 | g | 0.19 | oz | 0.006 | oz |
| Khus Khus | 5.5 | g | 0.19 | oz | 0.006 | oz |
| Athimaduram | 5.5 | g | 0.19 | oz | 0.006 | oz |
| Valampuri/Edampuri | 5.5 | g | 0.19 | oz | 0.006 | oz |
| Peppermint | 3.9 | g | 0.14 | oz | 0.005 | oz |
| Lavender | 3.9 | g | 0.14 | oz | 0.005 | oz |
| Chamomile | 3.9 | g | 0.14 | oz | 0.005 | oz |
| Thyme | 3.9 | g | 0.14 | oz | 0.005 | oz |
| Marigold | 3.9 | g | 0.14 | oz | 0.005 | oz |
| (Calendula) | | | | | | |
| Amla | 3.9 | g | 0.14 | oz | 0.005 | oz |
| Horsetail | 3.9 | g | 0.14 | oz | 0.005 | oz |
| Burdock | 3.9 | g | 0.14 | oz | 0.005 | oz |
| Moringa | 3.9 | g | 0.14 | oz | 0.005 | oz |
| Ginger | 3.9 | g | 0.14 | oz | 0.005 | oz |
| Comfrey | 3.9 | g | 0.14 | oz | 0.005 | oz |
| Ginkgo | 3.9 | g | 0.14 | oz | 0.005 | oz |
| Hibiscus | 3.9 | g | 0.14 | oz | 0.005 | oz |
| Stinging Nettle | 3.9 | g | 0.14 | oz | 0.005 | oz |
| Anise | 3.9 | g | 0.14 | oz | 0.005 | oz |

In this embodiment, the hair growth stimulant mixture is a combination of three parts by weight the combination of constituents as presented in Table 1 and between one part and nine parts weight 5% solution of 2,4-diamino-6-piperidinopyrimidine and well mixed to yield a homogeneous solution. The solution may be simmered for about an hour and then the resultant oil mixture strained, or left at room temperature for about three days and then the oil mixture strained. Alternately, all herbs are placed in a heat resistant (typically glass) container to which the following oils are added—grape seed, avocado, pumpkin seed oil, olive oil, and castor oil. The vessel is then immersed in boiling water for about 30-45 minutes, extracted from the water, and allowed to rest for about three weeks to extract all the nutrients from the herbs. The resultant oil mixture is then strained, such as through cheese cloth, and the biotin and optionally 2,4-diamino-6-piperidinopyrimidine is mixed with the resultant oil mixture to yield a homogeneous oil preparation ready for bottling.

Typically, the homogeneous oil preparation is 1:1 Table 1 constituents to 2,4-diamino-6-piperidinopyrimidine by weight. One or two drops of the resulting solution are applied topically to the affected area once or twice a day, being careful to not contact the oil with any portions of the skin where hair growth is unwanted. Hair regrowth should be observed within a few days.

In a third embodiment, the hair growth stimulant mixture is a combination of constituents as presented in Table 1 and well mixed to yield a homogeneous oil solution. The relative amounts of each are typically as presented in Table 1 above, although the amounts of one or more of the above-listed constituents may be varied by half to by double.

One or two drops of the resulting solution are applied topically to the affected area once or twice a day, being careful to not contact the oil with any portions of the skin where hair growth is unwanted. Hair regrowth should be observed within a few days.

In a fourth embodiment, the formulation is the same as that of the second and/or third embodiments, but with the addition of at least one part (0.14/0.15 oz.) tea tree oil.

In a fifth embodiment, rosemary oil, peppermint oil, biotin, aloe very, grapeseed oil, pumpkin seed oil, rice bran oil, avocado oil, castor oil, amla, neem, chebe powder, bhingraj, and onions are blended together to yield a homogeneous solution. The solution may be simmered for about an hour and then the oil strained, left at room temperature for about three days and then the oil strained, or treated as described above. The relative amounts of each are typically as presented in Table 1 above, although the amounts of one or more of the above-listed constituents may be varied by half to by double.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

What is claimed is:

1. A method for stimulating hair growth, comprising:
a) providing a homogeneous composition including five parts by weight 5% solution of 2,4-diamino-6-piperidinopyrimidine, one part by weight biotin, one part by weight rosemary oil, one part by weight tea tree oil, one part by weight peppermint oil and one part by weight pumpkinseed oil; and
b) topically applying the homogeneous composition to skin where hair is desired to grow.

2. The method of claim 1 wherein step b) is repeated twice daily until desired hair growth is achieved.

3. A hair regrowth composition comprising in combination:
five parts by weight 5% solution of 2,4-diamino-6-piperidinopyrimidine;
one part by weight biotin;
one part by weight rosemary oil;
one part by weight tea tree oil;
one part by weight peppermint oil; and
one part by weight pumpkinseed oil.

4. A hair regrowth composition comprising in combination:
one part by weight 5% solution of 2,4-diamino-6-piperidinopyrimidine; and
one part by weight admixture;
wherein the admixture further comprises:
one part by weight biotin;
one part by weight rosemary oil;
one part by weight tea tree oil;
one part by weight peppermint oil; and
one part by weight pumpkinseed oil.

5. The hair regrowth composition of claim 4 wherein the admixture further comprises:
sixty parts by weight biotin;
one-hundred parts by weight rosemary oil;
one part by weight tea tree oil;
one and a half parts by weight peppermint oil; and
one-hundred parts by weight pumpkinseed oil.

6. The hair regrowth composition of claim 5 wherein the admixture further comprises:
one-hundred parts by weight avocado oil;
one-hundred parts by weight bran oil;
one-hundred parts by weight castor oil;
one-hundred parts by weight olive oil;
one-hundred parts by weight grapeseed oil;
sixty parts by weight chebe powder;
four parts by weight rosemary;
four parts by weight aloe vera;
three parts by weight fenugreek;
three parts by weight rose petals;
two parts by weight neem;
two parts by weight bhringraj;
two parts by weight cloves;
two parts by weight curry leaves;
two parts by weight poolankilangu;
two parts by weight karboga arisi;
two parts by weight vembalam pattai;
two parts by weight kaddukai;
two parts by weight avarampoo;
two parts by weight maruthani vithai;
two parts by weight samangi vithai;
two parts by weight nellikai;
two parts by weight magalampoo;
two parts by weight avarakkai;
two parts by weight velankuchi ver;
two parts by weight onion;
two parts by weight karunjeeragam
two parts by weight vasambu;
two parts by weight khus khus;
two parts by weight athimaduram;
two parts by weight edampuri kai;
one and a half parts by weight lavender;
one and a half parts by weight chamomile;
one and a half parts by weight thyme;
one and a half parts by weight marigold;
one and a half parts by weight amla;
one and a half parts by weight horsetail;
one and a half parts by weight burdock;
one and a half parts by weight moringa;
one and a half parts by weight ginger;
one and a half parts by weight comfrey;
one and a half parts by weight ginkgo;
one and a half parts by weight hibiscus;
one and a half parts by weight stinging nettle; and
one and a half parts by weight anise.

7. The hair regrowth composition of claim 5 wherein the admixture further comprises:
one-hundred parts by weight avocado oil;
one-hundred parts by weight bran oil;
one-hundred parts by weight castor oil;
one-hundred parts by weight olive oil;
one-hundred parts by weight grapeseed oil;
sixty parts by weight chebe powder;
four parts by weight rosemary;
four parts by weight aloe vera;
three parts by weight fenugreek;
three parts by weight rose petals;
two parts by weight neem;
two parts by weight bhringraj;
two parts by weight cloves;
two parts by weight curry leaves;
two parts by weight pulankilangu;
two parts by weight karboga arisi;
two parts by weight vembalam pattai;
two parts by weight kaddukai;
two parts by weight avarampoo;
two parts by weight maruthani vithai;
two parts by weight samangi vithai;
two parts by weight nellikai;
two parts by weight magalampoo;
two parts by weight avarakkai;
two parts by weight velankuchi ver;
two parts by weight onion;
two parts by weight karunjeeragam;
two parts by weight vasambu;
two parts by weight khus khus;
two parts by weight athimaduram;
two parts by weight edampuri kai;
one and a half parts by weight lavender;
one and a half parts by weight chamomile;
one and a half parts by weight thyme;
one and a half parts by weight marigold;
one and a half parts by weight amla;
one and a half parts by weight horsetail;
one and a half parts by weight burdock;
one and a half parts by weight moringa;
one and a half parts by weight ginger;
one and a half parts by weight comfrey;
one and a half parts by weight ginkgo;
one and a half parts by weight hibiscus;
one and a half parts by weight stinging nettle; and
one and a half parts by weight anise.

8. A hair regrowth composition comprising in combination:
one part by weight biotin;
one part by weight chebe powder;
one part by weight rosemary oil;
one part by weight aloe vera;
one part by weight grapeseed oil;
one part by weight rice bran oil;
one part by weight avocado oil;
one part by weight castor oil;
one part by weight amla;
one part by weight neem;
one part by weight bhingraj;
one part by weight onions;
one part by weight peppermint oil; and
one part by weight pumpkinseed oil.
